(19) United States
(12) Patent Application Publication
(10) Pub. No.: US 2002/0103219 A1
(43) Pub. Date: Aug. 1, 2002

(54) Stable viscous liquid formulations of amlexanox for the prevention and treatment of mucosal diseases and disorders

(76) Inventor: Jeremy Jacob, Lewisville, TX (US)

Correspondence Address: JACKSON WALKER LLP, Suite 2100, 112 E. Pecan, San Antonio, TX, 78205-3731, US

(21) Appl. No.: 09/971,562

(22) Filed: Oct. 4, 2001

Related U.S. Application Data
(60) Provisional application No. 60/238,175, filed on Oct. 5, 2000.

(51) Int. Cl. A61K 9/14; A61K 31/4741

(52) U.S. Cl. 514/291; 424/487; 424/488

(57) ABSTRACT

Present invention concerns stable viscous liquid formulations of Amlexanox for the prevention and treatment of mucosal diseases and disorders. The mucoadhesive of the present invention may be a linear or cross-linked polymer such as polyacrylic acid, hydroxyalkylcellulose, dextran sulphate, and so forth. An object of the present invention is to provide a convenient and effective dosage form for Amlexanox in the treatment of mucocutaneous disorders. This form allows for an effective dose of the pharmaceutical to be applied to the lesions being treated over an extended period.

### BACKGROUND OF THE INVENTION

1. Field of the Invention

The present invention relates to compositions and methods of manufacture of stable, viscous, mucoadhesive liquid formulations of amlexanox, and the use of these compositions to coat mucocutaneous surfaces, such as those in the oral cavity, the nasal cavity, the gastrointestinal and respiratory tracts, the vagina, and the bladder, for the purpose of topical delivery of this pharmaceutical compound to prevent and treat ulcerative, inflammatory, and/or erosive, mucosal diseases and disorders. The liquid dosage forms are sufficiently mobile to coat a wide area of the mucosal surface, but also mucoadhesive and viscous, thus providing prolonged retention on the surface of the mucosa, enhancing the delivery of the pharmaceutically active compound to the surface of the mucosa, to enable effective prevention and treatment of diseases and disorders of mucosal surfaces.

2. Art Relevant to the Amlexanox Viscous, Mucoadhesive Solution Patent

A. Amlexanox Related Patents Include:

U.S. Pat. No. 4,143,042; U.S. Pat. No. 4,255,576; U.S. Pat. No. 4,299,963; U.S. Pat. No. 4,728,509, U.S. Pat. No. 5,632,737; and U.S. Pat. No. 5,952,338.

B. Amlexanox Related Publications Include:

Saijo et al.; Int Archs Alleroy Appl Immun, (1985a), 77, 315-321.

Saijo et al., Int Archs Allergy Appl Immun, (1985b), 78, 43-50.

Saijo et al., Int A2rchs Allergy Appl Immun, (1986), 79, 231-217.

Makino et al., Int. Archs Allergy appl Immun, (1987), 82, 66-71

C. Papers on Amlexanox Relating to Aphthous Ulcers Include:

Greer et al., J Oral Maxillofac Surg, (1993), 51(3):243-8.

Khandwala et al., Oral Sure Oral Med Oral Pathol Oral Radiol Endod, (1997a), 83(2):222-30.

Khandwala et al., Oral Surg Oral Med Oral Pathol Oral Radiol Endod. (1997b), 83(2):231-8.

Binnie et al., Compend Contin Educ Dent, (1997), 18(11), 1116-8, 1120-2, 1124.

D. Recent Meeting Abstracts Relating to Occular Administration of Amlexanox Solutions Include:

Ogawa et al., 69^{th} Annual Meeting of the Japanese Pharmacological Soc., Nagasaki, Japan, Mar. 20-23, (1996).

Watanabe et al., Annual Meeting of the Association for Research in Vision and Opthalmolmology, Fort Lauderdale, Fla., Apr. 21-26 (1996).

These two abstracts were cited in U.S. Pat. No. 5,952,338; the presentations describe the use of amlexanox solution in preclinical studies in a model of myopia.

C. Patents Relating to Oral Rinse Formulations Include:

U.S. Pat. No. 4,226,848; U.S. Pat. No. 5,011,852; U.S. Pat. No. 5,180,715; U.S. Pat. No. 5,310,545; U.S. Pat. No. 5,438,075; U.S. Pat. No. 5,945,087; U.S. Pat. No. 6,025,326; and PCT 00/04878.

D. Journal Publications Relating to Oral Rinse Formulations for Mucositis Include:

Dodd et al., Oral Surgery Oral Medicine Oral Pathology Oral Radiology & Endodontics., 2000, 90(1):39-47.

Turhal et al., Supportive Care in Cancer, 2000, 8(1):55-58.

Bez et al., Oral Surgery Oral Medicine Oral Pathology Oral Radiology & Endodontics, 1999, 88(3):311-315.

Fidler et al., Cancer, 1996, 77(3):522-525.

Mueller et al., American Journal of Health-System Pharmacy, 1995, 52(22):2596-2597.

E. Journal Publications Relating to Intra Vesical Treatment of Interstitial Cystitis Include:

Porru et al., Urol Int, 1997;59(1):26-9.

Parkin et al., Urology, 1997 49(5A Suppl):105-107.

Bade et al., Br J Urol, 1997, 79(2):168-171.

Mucous membranes provide a protective layer on the surface of several body cavities, such as the oral cavity, the nasal cavity, the gastrointestinal and respiratory tracts, the vagina, and the bladder. Cells within or glands adjacent to these membranes secrete mucus, a fluid or gel primarily composed of water, lipids, inorganic salts and mucin glycoproteins, which serve to form a protective barrier to inhibit passage of harmful materials to the underlying tissue. There are several diseases and disorders of these mucosal surfaces which can result in severe pain, irritation, edema, erythema, and/or ulceration. Examples of such diseases in the oral cavity include aphthous ulcers, oral lichen planus, and oral mucous membrane contact dermatitis; examples of diseases of the nasal mucous membrane include sinusitis and rhinitis; an example for the bladder is interstitial cystitis. Certain diseases such as Behget syndrome, graft-versus-host disease, and the pemphigoid group of diseases can affect the mucocutaneous membranes of several regions of the body: Many other ulcerative mucocutaneous diseases are known. Severe pain, irritation, edema, erythema, and/or ulceration of mucosal surfaces can also result as an adverse side-effect in certain therapies, such as chemotherapy and radiation therapy. Examples of such side-effects include mucositis, esophagitis, and radiation proctitis.

The following section provides a brief description of these conditions:

### Aphthous Ulcers

Aphthous ulcers (also known as aphthous stomatitis and canker sores) are benign open sores in the mouth, which appears as a painful white or yellow sore (ulcer) surrounded by a bright red area. Aphthous ulcers can be categorized into three groups:

(1) minor aphthous ulcers, the most common type, which recur in crops of 1 to 5 lesions, are less than 1 cm in diameter each, and usually affect the lips, buccal mucosa, mucobuccal and mucolabial sulci, and tongue;

(2) major aphthous ulcers, which are greater than 2 cm in diameter, begin as solitary nodules, and subsequently destroy deeper tissue, resulting in scarring that affects the movable oral mucosa and posterior mucosal surfaces; and

(3) herpetiform ulcers, which are recurrent, multiple (10 to 100), shallow, pinpoint lesions 1 to 2 mm in diameter that may affect any part of the mucosa. The cause for any of the three types is not known, although autoimmune mechanisms are suspected.

The cause is unknown. There may be an inherited predisposition to their development. There may also be an immune system link. Ulcers may develop in response to mouth injury such as dental procedures or aggressive tooth cleaning. They may occur at the site of a bite when the tongue or cheek is bitten. They can also be triggered by stress, dietary deficiencies (especially iron, folic acid, or vitamin B 12), menstrual periods, hormonal changes, food allergies, and similar situations: They may occur with no identifiable cause. Canker sores usually appear on nonkeratinized mouth tissue including the inner surface of the cheeks and lips, tongue, soft palate, and the base of the gums. They usually begin with a tingling or burning sensation, followed by a red spot or bump that ulcerates. Pain spontaneously decreases in 7 to 10 days, with complete healing in 1 to 3 weeks. Occasionally, a severe occurrence may be accompanied by nonspecific symptoms of illness such as fever or malaise. Recurrence is common and may continue for years (sources: WebMD, Rakel: Conn's Current Therapy 2000, 52nd ed).

Amlexanox is approved in a paste formulation by the U.S.A. Food and Drug Administration for the treatment of the signs and symptoms of minor aphthous ulcers.

Aphthous ulcers are occasionally associated with macrocytic anemias or gluten-sensitive enteropathy and may become more frequent and severe in association with human immunodeficiency virus (HIV) infection (source: Goldman: Cecil Textbook of Medicine, 21st Ed.).

### Behcet Syndrome

Behcet's disease is a chronic multisystem disease characterized by oral and genital aphthae, arthritis, cutaneous lesions, and ocular, gastrointestinal, and neurologic manifestations. It was first described by the Turkish dermatologist Hulusi Behcet in 1937 as “recurrent oral aphthous ulcers, genital ulcers, and ‘hypopyon-uveitis.’” The diagnosis of Behcet's disease is based on clinical criteria as established by O'Duffy and Goldstein and the International Study Group. Complex aphthosis is the presence of almost constant, multiple oral or oral and genital aphthae in the absence of systemic manifestations. These patients must be distinguished from those with Behcet's disease.

The prevalence of Behcet's disease is higher in the Middle East and Japan where it offsets approximately 1 in 1000. The disease is far less common in northern Europe, the United States, and the United Kingdom. The mean age of onset ranges from the mid to late 20s to the fourth decade, according to several series, with a slightly higher male to female ratio. It is relatively rare in children and the elderly. Behcet's disease is also uncommon among black Africans who, when they are affected, tend to have more mucocutaneous features. Although a definitive pattern of inheritance has not been elucidated, familial cases have been reported. Patients with complex aphthosis are probably a subset of patients with recurrent aphthous stomatitis, which is defined as the recurrence of one or more painful oral ulcers at intervals ranging from days to months. The prevalence of recurrent aphthosis ranges from 5% to 66%. Onset may occur in childhood or adolescence and some patients experience a decrease in frequency with advancing age (source: J. V Ghate and J. L. Jorizzo, “Behcet's disease and complex aphthosis”, Journal of the American Academy of Dermatology, 1999, 40(1), 1-18.).

### Chemical and Radiation Cystitis

The term ‘cystitis’ covers a range of disorders commonly known “painful bladder” disease in which sufferers have bladder and/or pelvic pain and irritative voiding symptoms, (urgency, frequency, nocturia, dysuria). There are a variety of known causes, which includes the damaging side-effects of radiation therapy to the lower abdomen, and cytotoxic agents and/or their metabolites as they pass through the bladder following renal clearance.

Cyclophosphamide (Cytoxan), the most commonly used oxazaphosphorine, is an alkylating agent first used in 1957 for treatment of malignant tumors in Europe in 1957. Cyclophosphamide still has a role in the treatment of solid tumors and lymphomas, as well as benign inflammatory states, Wegener's granulomatosis and rheumatoid arthritis being the most common. Other oxazaphosphorines—ifosfamide, trofosphamide, and sufosfamide—have been used since the 1970s for the treatment of solid malignancies and lymphomas. Dose-limiting toxicity with these compounds is usually urinary tract toxicity.

Subsequent to treatment with these compounds, urinary symptoms including frequency, urgency, dysuria, and nocturia develop in as many as 24% of patients treated with oral Cytoxan.

Bladder pathology has been attributed to toxic metabolites of these compounds. Cyclophosphamide is broken down by hepatic microsomal cells to hydroxycyclophosphamide; then by target cells to aldophosphamide; and then to phosphoramide mustard, the active antineoplastic metabolite, and acrolein, which has no significant antitumor activity. Similarly, ifosfamide is metabolized to iphosphoramide mustard and acrolein. Urinary excretion of acrolein is believed to be the major source of urothelial toxicity. Most normal cells are able to break down the toxic metabolites and diminish their effect. Glutathione is a naturally occurring thiol that can confer such protection in most cells, but is present in low levels in urine.

Oxazaphosphorine toxicity has been demonstrated in several animal models with their systemic administration and by installation of their normal metabolic products directly into the bladder. Urine from animals given these agents, when placed in other animal bladders, will reproduce these findings, while instillation of cyclophosphamide will not. Electron microscopy suggests the initial toxic effect is disruption of the plasma membrane and cytoplasmic matrix.

Bladder damage from these compounds is cumulative, and is generally dose related. “Cyclophosphamide cystitis” occurs frequently and early following intravenous (IV) therapy, especially dose-intensive regimens. Cystitis usually takes weeks to develop after oral treatment, but has been seen after as little as one dose. Fibrosis has been found in as many as 25% of children receiving high-dose cyclophosphamide. Severe hematuria and telangiectasia are more common in these patients. Oxazaphosphorine cystitis is potentiated by prior pelvic radiation.

Cystoscopy may reveal a tumor or changes compatible with cyclophosphamide cystitis. Acutely diffuse inflammation is seen. Chronic changes include a pale bladder mucosa with telangiectasia. Areas of edema can be present with patchy hemorrhagic areas that stain with methylene blue, an indicator of mucosal injury. Biopsies reveal hyperemia, hemorrhage, edema, mucosal thinning, and ulceration of the urothelium. Necrosis of mucosa, muscle, and small arterioles and telangiectasia can be present. Atypia can be prominent, and abundant mitoses often occur. These findings are similar to those seen after radiation therapy (source: DeVita: Cancer: Principles and Practice of Oncology, 5th ed., Copyright© 1997 Lippincott-Raven Publishers).

### Erythema Multiforme

Erythema multiforme often affects the oral cavity and is frequently recurrent. The classic cutaneous findings are targetoid lesions symmetrically distributed over the trunk and extremities. Studies show that as many as 70% of patients develop oral lesions, which are extremely painful and often debilitating. In more than 60% of patients, the attacks followed an episode of herpes simplex virus (HSV) infection. Acyclovir or one of the newer antiviral agents can be used to suppress recurrent HSV outbreaks and to prevent recurrent erythema multiforme. The current recommendations for daily suppression of HSV are acyclovir (400 mg twice daily), famciclovir (250 mg twice daily), or alacyclovir (500 mg daily). Suppressive doses are to be used for patients who experience more than six episodes a year of HSV or HSV-induced erythema multiforme.

Azathioprine (Imuran) can also be used to treat erythema multiforme. It should be used for patients who have severe mucosal involvement or in whom lesions continue to occur despite HSV suppression. In one study, 11 patients with severe disease failed to respond to acyclovir, dapsone, or antimalarials, but all cleared with azathioprine, 100 to 150 mg daily. Response was dose-dependent, and the condition relapsed when therapy was discontinued. Thalidomide has also been shown to be effective for recurrent erythema multiforme (source: J. L. Popovsky and C. Camisa, “New and emerging therapies for diseases of the oral cavity”, Dermatologic Clinics, 2000, 18(1), 113-125).

### Esophagitis

Esophagitis is a side-effect of the radiation therapy of the chest when the esophagus receives an unavoidable significant radiation dose. Acute side effects occurring during the course of radiotherapy are organ specific and related to the fractionation scheme, total dose, and use of sequential or concomitant chemotherapy or radiosensitizers. They typically manifest in the second to third week of treatment. A significant concern of combined therapy is the increased toxicity, which may potentially outweigh the benefit from both modalities.

Most patients do not develop symptoms related to lung irradiation until the end of treatment. Some patients complain of a dry, nonproductive cough secondary to radiation effect on the trachea or bronchi. This reaction can last for several weeks after the completion of treatment. Radiation may also induce acute esophagitis during the course of therapy, which typically occurs during the second through fourth weeks of treatment, as well as shortly after completion. Chemotherapy and radiosensitizers appear to accelerate the onset and severity of symptoms. Agents such as 5-fluorouracil, doxorubicin, cisplatin, and mitomycin enhance the effect of radiation with regard to the esophagus. In general, acute esophagitis resolves shortly after the completion of radiotherapy, with few patients progressing to chronic esophagitis. Esophagitis presents with mild to severe swallowing difficulty requiring diet modification and nonnarcotic or narcotic analgesics, depending on severity (source: DeVita: Cancer: Principles and Practice of Oncology, 5th ed.).

### Graft-Versus-Host Disease (GVHD)

GVHD occurs when genetically disparate lymphocytes are transferred into an immunologically compromised recipient incapable of rejecting the donor graft . The initial stages of a graft-versus-host (GVH) reaction involve the recognition of the host's disparate minor and major histocompatibility antigens by T cells in the donor graft. Major histocompatibility antigens (MHC) are primarily of two classes:

1) HLA class I antigens have abroad distribution and are expressed on all cells;

2) HLA class II antigens are expressed on antigen-presenting cells, including macrophages, dendritic cells, B cells, and activated T cells.

Minor histocompatibility antigens (mHC) represent genetic polymorphisms of endogenous cellular proteins which are presented to T cells as small peptides bound in the grooves of the major histocompatibility antigens.

Acute GVHD involves three organ systems primarily—the skin, the gastrointestinal tract, and the liver. A maculopapular rash is often present and can be pruritic or painful. It can involve the palms and soles. In severe cases bullae and epidermal separation may occur which resemble toxic epidermal necrolysis. Gastrointestinal GVHD involves the small and large intestine and may result in nausea or vomiting, crampy abdominal pain, diarrhea, intestinal bleeding, and ileus. More recently, a variant of GVHD with symptoms of persistent anorexia, nausea, and vomiting has been recognized. Lesions in the mouth may also occur. Cholestatic jaundice is common in liver GVHD but hepatic failure caused solely by GVHD of the liver is unusual unless the GVHD is long-standing. A biopsy of these organs is useful for confirming diagnosis of GVHD, however, direct cytotoxic effects from the conditioning regimen can produce tissue damage which may confound the histopathologic diagnosis of GVHD in the first 3 weeks after marrow transplantation.

Chronic GVHD is a syndrome that may develop as early as 50 to 60 days or as late as 400 days after transplantation. The incidence of chronic GVHD is dependent on the degree of disparity in the major histocompatibility antigens. It was observed in 33% of HLA-identical sibling transplants, 49% of HLA-nonidentical related transplants, and 64% of matched-unrelated transplants. A prior history of acute GVHD is a significant risk factor for the development of chronic GVHD. Among patients without previous acute GVHD, factors associated with an increased risk of de novo chronic GVHD were increasing patient age, the. infusion of buffy coat cells in addition to marrow, and corticosteroid therapy before transplantation. Corticosteroid use for GVHD prophylaxis may also increase the risk of the development of chronic GVHD.

Clinical features of chronic GVHD are skin lesions which may initially resemble lichen planus and may progress to generalized scleroderma, keratoconjunctivitis, buccal mucositis, esophageal and vaginal strictures, intestinal abnormalities, chronic liver disease, pulmonary insufficiency secondary to bronchiolitis obliterans, and a wasting syndrome. If generalized scleroderma occurs, it may lead to joint contractures and debility; however this feature of chronic GVHD has not been seen as commonly in recent years. Elevations in alkaline phosphatase and serum bilirubin are often the first indication of hepatic involvement with chronic GVHD. Bile duct damage has a similar histopathology as that seen in primary biliary cirrhosis. Liver biopsies are often helpful in establishing a diagnosis. Onset of bronchiolitis obliterans may occur at less than 150 days (40%) or more than 150 days (60%) following HSCT. Response to immunosuppressive treatment has been observed but is often incomplete. Prognosis is poor except for those patients with mild to moderate disease occurring more than 150 days following HSCT. Keratoconjunctivitis sicca is a common complication and is generally irreversible. This is managed by frequent eye drops and tear duct ligation. Screening studies and clinical manifestations of chronic GVHD are used in a clinical classification of chronic GVHD. Patients with limited disease involving only the skin or the liver have a favorable course even if untreated, compared with patients with multiple organ involvement. (Source: Lee: Wintrobe's Clinical Hematology, 10th ed.)

### Interstitial Cystitis

Interstitial cystitis (IC) has only recently been recognized as a major health problem. It encompasses a major portion of the “painful bladder” disease complex, which includes a large group of urologic patients with bladder and/or pelvic pain, irritative voiding symptoms (urgency, frequency, nocturia, dysuria), and negative urine cultures. Painful bladder diseases with a well-known cause include radiation cystitis, cyclophosphamide cystitis, cystitis caused by microorganisms that are not detected by routine culture methodology, and systemic diseases affecting the bladder.

One problem with defining IC is that the symptoms are in reality an exaggeration of normal sensations. Urinary frequency patterns can be related to fluid intake and age, and the signal or urge to void is considered an unpleasant or painful sensation by most persons. With no pathognomonic findings on pathologic examination, IC is truly a diagnosis of exclusion. It may have multiple causes and represent a final common reaction of the bladder to different types of insult. Thus, issues of definition are critical. To understand the current way IC is defined and how this came to be, a look back in time is helpful.

In 1987, there were 43,500 (perhaps up to 90,000) diagnosed cases of IC in the United States, approximately twice the prevalence in Finland found by Oravisto 12 years earlier. More interesting, women who were diagnosed by sampled urologists as actually having IC represented only 20% of the cases presenting with symptoms (chronic painful bladder, sterile urine) that were suggestive of this disease. On the basis of these data, from 250,000 to almost 500,000 patients in the United States might have had IC in 1987, depending on the assumptions used.

The median age of onset is 40 years. Up to 50% of patients experience spontaneous remissions probably unrelated to treatment with a duration ranging from 1 to 80 months (mean 8 months). Patients with IC are 10 to 12 times more likely than controls to report childhood bladder problems. The time from symptom onset to diagnosis varied from 24 months for the patients most recently diagnosed to 51 months for members of the Interstitial Cystitis Association. Taking the prevalence figure of 44,000, the IC-related incremental medical care cost in the United States was $116.6 million in 1987 and IC-related lost economic production was $311.7 million.

Intra vesical lavage with one of a variety of preparations remains the standard treatment against which other treatments must be measured. A mainstay of the treatment of IC is the intra vesical instillation of DMSO. DMSO is a product of the wood pulp industry and a derivative of lignin. It has exceptional solvent properties and is freely miscible with water, lipids, and organic agents. Pharmacologic properties include membrane penetration, enhanced drug absorption, anti-inflammatory properties, analgesic properties, collagen dissolution, muscle relaxation, and mast cell histamine release. In vitro effects on bladder function belie its positive effects in vivo (source: Walsh: Campbell's Urology, 7th ed., Copyright© 1998 W. B. Saunders Company).

### Mucositis

Oral mucositis is a significant problem in patients receiving chemotherapy or radiation therapy. Estimates of oral mucositis in cancer therapy range from 40% of those receiving standard chemotherapy to 76% of bone marrow transplant patients. Virtually all patients who receive radiation therapy to the head and neck area develop oral complications. Mucositis is not only painful, but it also can limit adequate nutritional intake and decrease the willingness of patients to continue treatment. More severe mucositis with extensive ulceration may require costly hospitalizations with parenteral nutrition and narcotics. Mucositis diminishes the quality of life and may result in serious clinical complications. A healthy oral mucosa serves to clear microorganisms and provides a chemical barrier that limits penetration of many compounds into the epithelium. A mucosal surface that is damaged increases the risk of a secondary infection and may even prove to be a nidus for systemic infection. Mucositis may result in the need to reduce dosage in subsequent chemotherapy cycles. or to delay radiation therapy, which may ultimately affect patient response to therapy.

Normally, cells of the mouth undergo rapid renewal over a 7- to 14-day cycle. Both chemotherapy and radiation therapy interfere with cellular mitosis and reduce the ability of the oral mucosa to regenerate. Cancer chemotherapeutic drugs that produce direct stomatotoxicity include the alkylating agents, antimetabolites, natural products, and other synthetic agents such as hydroxyurea and procarbazine hydrochloride. Typical sequelae of these cytotoxic agents include epithelial hyperplasia, collagen and glandular degeneration, and epithelial dysplasia. Mucositis is an inevitable side effect of radiation. The severity of the mucositis is dependent on the type of ionizing radiation, the volume of irradiated tissue, the dose per day, and the cumulative dose. As the mucositis becomes more severe, pseudomembranes and ulcerations develop. Poor nutritional status further interferes with mucosal regeneration by decreasing cellular migration and renewal.

Direct stomatotoxicity is usually seen 5 to 7 days after the administration of chemotherapy or radiation therapy. In the nonmyelosuppressed patient, oral lesions heal within 2 to 3 weeks. The nonkeratinized mucosa is most affected. The most common sites include the labial, buccal, and soft palate mucosa, as well as the floor of the mouth and the ventral surface of the tongue. Clinically, mucositis presents with multiple complex symptoms. It begins with asymptomatic redness and erythema and progresses through solitary white elevated desquamative patches that are slightly painful to contact pressure. Following this large, acutely painful contiguous pseudomembranous lesions will develop with associated dysphagia and decreased oral intake. Histopathologically, edema of the retepegs is noted, along with vascular changes that demonstrate a thickening of the tunica intima with concomitant reduction in the size of the lumen and destruction of the elastic and muscle fibers of the vessel walls. The loss of the epithelial cells to the basement membrane exposes the underlying connective tissue stroma with its associated innervation, which, as the mucosal lesions enlarge, contributes to increasing pain. Oral infections, which may be due to bacteria, viruses, or fungal organisms, can further exacerbate the mucositis as well as lead to systemic infections. If the patient develops both severe mucositis and thrombocytopenia, oral bleeding may occur that is very difficult to treat.

A mucositis grading system gives the physician the ability to assess the severity of the mucositis in terms of both the pain and the patient's ability to maintain adequate nutrition so that a treatment plan can be appropriately constructed. There are many different grading systems; most are based on two or more clinical parameters, including erythema, pain, and problems with eating. An example of a common grading system is that proposed by the National Cancer Institute, which uses a numbering scale of 0 to 4. Grade 0 means no mucositis; grade 1, the patient has painless ulcers, erythema, or mild soreness; grade 2, the patient has painful erythema, edema, or ulcers but can eat; grade 3, the patient has painful erythema, edema, or ulcers and cannot eat; and grade 4, the patient requires parenteral or enteral support.

A standardized approach for the prevention and treatment of chemotherapy- and radiation-induced mucositis is essential; unfortunately, the efficacy and safety of most of the regimens have not been established. The prophylactic measures usually employed for the prevention of mucositis include chlorhexidine gluconate (Peridex), saline rinses, sodium bicarbonate rinses, acyclovir, amphotericin, and ice. Regimens commonly used for the treatment of mucositis and its associated pain include a local anesthetic such as lidocaine or Dyclone, Maalox or Mylanta, diphenhydramine (Benadryl), nystatin, or sucralfate. These agents are either used alone or in different combinations of the above medications made into a mouthwash. Other agents used less commonly include Kaopectate, allopurinol, vitamin E, beta-carotene, Kamillosan liquid, aspirin, antiprostaglandins, prostaglandins, MGI 209 (marketed as Oratect Gel), silver nitrate, and antibiotics. Oral and sometimes parenteral narcotics are used for pain relief. A new method utilizing capsaicin is currently under study to help relieve the pain (source: DeVita: Cancer: Principles and Practice of Oncology, 5th ed., Copyright© 1997 Lippincott-Raven Publishers).

### Oral Lichen Planus

Lichen planus (LP) is a common, idiopathic skin disorder affecting approximately 2% of the adult US population. Although its behavior on the skin is predictable and manageable using topical corticosteroids, oral lichen planus (OLP) has a more variable clinical course and is less responsive to topical corticosteroid therapy. There are multiple clinical presentations of OLP, and the disorders in some of these clinical forms can mimic many other types of oral lesions. Furthermore, some authors believe that certain clinical types of OLP may have a premalignant nature. Various drugs, topical and systemic, have been shown to induce lichenoid lesions through antigenic mechanisms.

There is even good evidence emerging that amalgam and dental plaque can act as antigens to induce OLP in some patients. The plaque form of OLP is seen more often in smokers. Women appear to be affected more often than men. Not all persons who develop skin lesions develop OLP at the same time and vice versa. Those patients who develop skin lesions only are usually free of their LP in approximately 18 months; however, patients with OLP may have their lesions for up to 20 years. Thus, management strategies for the patient with OLP are markedly different than for its skin surface counterpart.

The most common oral presentation of LP is the reticular form. These lesions appear as raised white, linear striations that often interlace in what is termed striae of Wickham. These striations are almost pathognomonic of the disorder. It should be noted that these linear lesions also accompany the erosive form of OLP and occur at the periphery of the eroded area. This is a significant diagnostic clue in the evaluation of the erosive type. The reticular form usually is observed on the buccal mucosa, often bilaterally. Several authors have noted that these lesions are adjacent to gold or silver amalgam restorations in many cases. The lesions are asymptomatic.

The bullous form of OLP is uncommon, perhaps because the oral cavity is a very active region. The functions of chewing, swallowing, and speaking probably do not allow the bulla to remain intact for very long. The size of these lesions is variable, from a few millimeters to several centimeters. The plaque type of OLP appears as a nondescript leukoplakia that needs to be biopsied if no other diagnosis can be made for the lesion. These lesions appear as multiple diffuse, raised white plaques commonly on the buccal mucosa and tongue. Silverman et al have determined that patients with this form of LP tend to be smokers. This may place them at risk for transformation to dysplasia or carcinomatous change.

The atrophic form of OLP can be seen concomitantly with the erosive or reticular forms. This is frequently the type of OLP seen on the gingiva of patients, commonly referred to as desquamative gingivitis. These lesions are symptomatic. The patient may complain of burning and pain while brushing. Because dental plaque has been implicated as a possible antigen, the patient will need to see a dentist for professional maintenance following initial corticosteroid treatment of the lesions.

Erosive LP is the most painful form of OLP. As stated previously, these erosions are seen frequently with the reticular form adjacent to the area. Atrophic or plaque forms may be seen less commonly. Erosive LP may mimic oral cancer, erythema multiforme, lupus erythematosus, and candidiasis. Many drugs can produce lesions that look-like erosive LP clinically (source: D. A. Miles and M. M. Howard, “Disorders affecting the oral cavity: Diagnosis and management of oral lichen planus”, Dermatologic Clinics, 1996, 14(2), 281-290).

### The Pemphigoid Group of Diseases

Bullous pemphigoid is a rare, relatively benign subepidermal blistering disease of unknown origin in which IgG autoantibodies are found both circulating and bound in the lamina lucida region of the basement membrane zone of the epidermis. There is no racial or gender prevalence. Pemphigoid is a disease of the elderly, with most cases occurring after age 60, although cases have been reported in children. There have been many reports of the coexistence of bullous pemphigoid with other disorders, but their association is probably coincidental. Drugs are often suspected of causing pemphigoid; and stopping medication or changing to a different oral medication may help. Oral blisters, if present, are mild and transient. Pemphigoid begins with a localized area of erythema or with pruritic urticarial plaques that gradually become more edematous and extensive.

Cicatricial pemphigoid (or benign mucous membrane pemphigoid) is a rare, chronic, subepidermal blistering and scarring disease disease seen in the elderly. The oral cavity and the eye are most frequently involved. Unlike bullous pemphigoid, there are few remissions. The mouth is involved in 85% of cases. Desquamative gingivitis is the most frequent manifestation. The gingiva appears red with diffuse or patchy involvement. Oral vesiculobullous lesions form, then rupture, leaving clean, noninflamed erosions that are relatively painless and do not interfere with eating. The vermilion border of the lips is spared, in contrast to the situation with pemphigus. Hoarseness is a sign of laryngeal involvement (8% of cases). The eye is involved in 65% of cases. Unilateral conjunctivitis is often the initial presentation; within 2 years the disease is usually bilateral. Fibrosis beneath the conjunctival epithelium is the primary destructive process. Gradual shrinkage of the conjunctiva leads to obliteration of the conjunctival sac. Reduced tearing with erosion and neovascularization of the cornea leads to corneal opacification and perforation. Fibrous conjunctival adhesions become more numerous; the disease leads to blindness in approximately 20% of cases. Patients with only ocular lesions are classified as having ocular cicatricial pemphigoid. These patients have lower in vivo deposits of IgG and C3, higher deposits of fibrin, absence of circulating antibodies, and negative serologic reactivity to bullous pemphigoid antigens. Severe ocularmucosal injury, such as that which occurs in Stevens-Johnson syndrome, may be a precipitating factor in the development of ocular cicatricial pemphigoid. The time lag between the onset of Stevens-Johnson syndrome and cicatricial pemphigoid ranged from a few months to 31 years. Prolonged periods of remission after stopping therapy occur in one third of patients. Follow-up must be continued for life, because relapse occurs in 22% of those who were in remission and not undergoing therapy. (Source: Habif: Clinical Dermatology, 3rd ed.)

### Pemphigus

Pemphigus is a rare, autoimmune blistering disease. Of the various forms of pemphigus, pemphigus vulgaris and paraneoplastic pemphigus affect the oral mucosa with regularity. The antigenic targets in pemphigus are components of the desmosome. Binding of autoantibodies to these antigenic proteins, desmogleins and desmoplakins, leads to dissolution of intercellular adhesion with resultant blister formation. Activity of the disease correlates with titers of pemphigus antibody, which can be detected in the serum of patients with the disease by indirect immunofluorescence testing.

Pemphigus was often a fatal disease before the use of systemic corticosteroids. It has a chronic course, and control of the disease becomes more difficult with subsequent flares. The basis of treatment is immunosuppression to decrease antibody synthesis. Relapses may occur when immunosuppressive drugs are tapered. Paraneoplastic pemphigus remains a very difficult disease to treat and continues to have very high mortality rates.

Historically, therapy for pemphigus was initiated with very high doses of prednisone. This initial high dosage proved to have so many adverse effects that in one early study 8% of patients died from complications of treatment (mainly infections) rather than from pemphigus. Pemphigus may initially be treated with prednisone, 60 to 80 mg daily, and generally no more than 100 mg daily. Studies have shown that this dosage is usually as effective as higher doses. If there is no response, but the patient remains stable, the authors recommend adding a second immunosuppressive agent rather than increasing the dose of steroids. Although there are no good controlled studies to support this practice, it is also being followed in the treatment of organ transplant rejection to decrease morbidity from corticosteroids. In patients in whom the disease must be rapidly controlled, higher doses of prednisone may be required to stabilize the patient.(source: J. L. Popovsky and C. Camisa, “New and emerging therapies for diseases of the oral cavity”, Dermatologic Clinics, 2000, 18(1), 113-125).

### Radiation Proctitis

Acute complications of pelvic radiation occur with distinct clinical courses and pathologic manifestations. The most frequent serious complication of pelvic radiation is small bowel damage, including thrombocytopenia, leukopenia, dysuria, and effects on the small bowel (diarrhea, abdominal cramping, and increased bowel frequency) and large bowel (acute proctitis, tenesmus, bloody and/or mucus discharge). Sigmoidoscopy during treatment normally reveals an inflamed, edematous, and friable rectal mucosa consistent with acute radiation proctitis. These symptoms are usually transient and resolve within a few weeks following the completion of radiation therapy. They appear to be a function of the dose rate and fraction size rather than the total dose. The mechanism is primarily the depletion of actively dividing cells in what is otherwise a stable cell renewal system. In the small bowel, loss of the mucosal cells results in malabsorption of various substances including fat, carbohydrate, protein, and bile salts. The management of bowel-related complications usually involves the use of diphenoxylate and/or narcotics. The bowel mucosa usually recovers in 1 to 3 months following the completion of radiation (source: DeVita: Cancer: Principles and Practice of Oncology, 5th ed., Copyright© 1997 Lippincott-Raven Publishers).

### Ulcerative Colitis

Inflammatory bowel diseases (IBD), including ulcerative colitis and Crohn's disease, are chronic inflammatory diseases of the gastrointestinal tract. They are diagnosed by a sea of clinical, endoscopic, and histologic characteristics, but no single finding is absolutely diagnostic for one disease or the other. Moreover, some patients have a clinical picture that falls between the two diseases and are said to have indeterminate colitis.

The inflammatory response in ulcerative colitis is largely confined to the mucosa and submucosa, but in Crohn's disease the inflammation extends through the intestinal wall from mucosa to serosa. Ulcerative colitis is confined to the colon, and colectomy is a curative procedure. Crohn's disease, in contrast, can involve any part of the gastrointestinal tract, although the distal small bowel and the colon are most commonly involved. Resection of the inflamed segment is not curative in Crohn's disease, and inflammation is likely to recur.

In ulcerative colitis, inflammation begins in the rectum, extends proximally a certain distance, and then abruptly stops, with a clear demarcation between involved and uninvolved mucosa. In mild disease, there are superficial erosions, whereas in more severe disease, ulcers may be large but superficial, penetrating the muscularis mucosa only in very severe disease. Inflammatory polyps or pseudopolyps may be present. Most of the pathologic findings in ulcerative colitis are limited to the mucosa and submucosa; the muscularis propria is affected only in fulminant disease. Active ulcerative colitis. is marked by neutrophils in the mucosa and submucosa and clumps of neutrophils in crypt lumens (crypt abscesses). There is mucus depletion, mucosal edema, and vascular congestion with focal hemorrhage. In addition to signs of acute activity, there are also signs of chronicity, with lymphoid aggregates, plasma cells, mast cells, and eosinophils in the lamina propria.

The dominant symptom in ulcerative colitis is diarrhea, which is usually associated with blood in the stool. Bowel movements are frequent but small in volume as a result of irritability of the inflamed rectum. Urgency and fecal incontinence may limit the patient's ability to function in society. Other symptoms include fever and pain, which may be in either lower quadrant or in the rectum. Systemic features—fever, malaise, and weight loss—are more common if all or most of the colon is involved and may have a greater effect than diarrhea on the patient's ability to function. Some patients, especially elderly persons, complain of constipation rather than diarrhea because rectal spasm prevents the passage of stool. The initial attack of ulcerative colitis may be fulminant with bloody diarrhea, but more commonly the disease begins indolently, with non-bloody diarrhea progressing to bloody diarrhea. Ulcerative colitis can present initially with any extent of anatomic involvement, from disease confined to the rectum to pancolitis. Most commonly, ulcerative colitis follows a chronic intermittent course with long periods of quiescence interspersed with acute attacks lasting weeks to months; however, a significant percentage of patients suffer a chronic continuous course. (source: Goldman: Cecil Textbook of Medicine, 21st Ed)

In the diseases and disorders listed above, there are either no adequate treatment options, or the treatments, while demonstrating success, may lead to the development of significant adverse side-effects. The physiology of these diseases and disorders are such that significant patient benefit could be derived from a safe pharmaceutical agent which possesses the ability to modulate several biochemical pathways which lead to the development of inflammatory and allergic responses.

Amlexanox (2-amino-7-(1 methylethyl)-5-oxo-5H[1]benzopyrano[2,3-b]pyridine-3-carboxylic acid) is a compound known to have anti-allergic and anti-inflammatory activities (table 1) which can be attributed to the compound's diverse pharmacological profile. Amlexanox has been shown to have antiallergic activity, to inhibit bronchoconstriction, and to have some anti-inflammatory effects in models for both chronic and acute inflammation. Studies of the mechanism of action of amlexanox, both in vivo and in vitro, have indicated that the agent has the following mechanisms of action:

1. Inhibition of the immunologically-stimulated release of histamine from mast cells.

2. Inhibition of leukotriene D4 (LTD4) generation.

3. Antagonism of the contractile response of airway smooth muscle to LTD4.

The antiallergic action of amlexanox was studied using various models for Type I to Type IV allergic reactions. The drug exerted a potent inhibitory effect on rat passive cutaneous anaphylaxis mediated by IgE (Type I). The intravenous and oral ED50 values were 0.032 and 0.73 mg/kg, respectively. Amlexanox inhibited the Arthus reaction in guinea pigs (Type III) with moderate activity at intravenous doses of5 and 20 mg/kg given immediately after antigen challenge and moderate activity at oral doses of 50 and 10 mg/kg given 1 or 3 hours after antigen challenge. Intravenous amlexanox inhibited the tuberculin reaction and 2,4-dinitro-1 -fluorobenzene-induced contact sensitivity in mice (Type IV). For both tests, significant inhibition was observed at 20 mg/kg, but not at 5 mg/kg.

Amlexanox, either at an intravenous dose of 1 mg/kg or an oral dose of 10 mg/kg, significantly suppressed IgE-mediated bronchoconstriction in rats. For actively sensitized guinea pigs, an ED50 value of 14.5 mg/kg was determined for inhibition of. IgE-mediated constriction in one study and significant inhibition was observed at 20 mg/kg in another study. For IgG1-mediated bronchoconstriction in actively sensitized guinea pigs, the ED50 values were 8.2 and 160 mg/kg for intravenous and oral administration of amlexanox, respectively. For guinea pigs passively sensitized with rabbit IgG, the ED50 value was 9 mg/kg, intravenously, for inhibition of bronchoconstriction.

Amlexanox, at intravenous doses of 5 and 20 mg/kg, showed dose-related suppression of the bronchoconstriction in guinea pigs induced by leukotriene D4 and platelet activating factor. The drug slightly suppressed histamine-induced bronchoconstriction and was not active against acetylcholine-induced bronchoconstriction.

Amlexanox in oral tablets (25 and 50 mg) is marketed in Japan by Takeda Pharmaceuticals for the treatment of bronchial asthma (approved in 1987) and allergic rhinitis (approved in 1989). Amlexanox in a nasal solution of 0.25% is also marketed by Takeda Pharmaceuticals in Japan for the treatment of allergic rhinitis (approved in 1988), and amlexanox in a ophthalmic solution of 0.25% is marketed by Senju Pharmaceuticals in Japan for the treatment of allergic conjunctivitis (approved in 1989). Amlexanox also exists as a 5% oral paste formulation which was approved by the U.S. Food and Drug Administration for the treatment of the signs and symptoms of minor aphthous ulcers in 1995.

U.S. Pat. No. 5,362,737 describes the use of amlexanox for the treatment of aphthous ulcers, and unspecified mucocutaneous disorders utilizing such dosage forms as an oral paste, solution, gel, quick-disintegrating tablet, mouthwash, ointment, cream, powder, adhesive patch, aerosolized spray, lozenze, troche, dentifrice, or dental floss. Of these possibilities, U.S. Pat. No. 5, 632,737 indicates that paste, lozenge, troche, solution, and gel were preferred due to the relative ease with which these formulations may be applied to the lesion with a finger tip, or the ease with which they can be masticated for contact with the oral mucosa.

The advantages of a mouthwash formulation are given as “its ability to reach deeper crevices between teeth and the distant areas of the mouth which are inaccessible to the fingertips for a comfortable or convenient mode of application”. Examples are given of mouthwash formulations containing water (60-95% w/w), ethanol (8-15%), glycerin (1.5-6.5%), sodium citrate (0.1-0.9%), triethanolamine (0.1-0.5%) and amlexanox (0.1-1.5%).

The mouthwash formulations described in U.S. Patent 5,362,737 have two possible disadvantages: lack of viscosity or mucoadhesion, and the potential to cause irritation or pain to the lesion because of the ethanol in the product. While standard oral rinse formulations, such as that described above, provide a convenient method for bringing an active pharmaceutical compound into contact with all or part of the oral cavity, subjects usually find it possible to retain a rinse solution in the mouth for only a brief period. Once the solution is either swallowed or expelled, the majority of the active ingredient is removed from the region where it is required to provide benefit. Any residual rinse is quickly washed out of the oral cavity by normal saliva flow. As a result, standard oral rinse formulations can only be used in conjunction with fast-acting active pharmaceutical compounds and are unsuitable for use with active pharmaceutical compounds which are most effective when they need to be in contact with the oral mucosa for an extended period.

A number of different approaches are known in the art to increase the amount of time an active pharmaceutical compound is in contact with the oral mucosa and hence increase the therapeutic effectiveness of an active pharmaceutical compound. Such approaches include the use of gels, pastes, tablets, and films. These compositions may or may not utilize mucoadhesive components to retain the dosage form in a single location. None of these solid or semi-solid dosage forms are convenient for use in treating lesions which are not readily accessible to application using fingertips, or in widespread disease within the oral cavity. By comparison, a liquid dosage form can be used to cover a wide area of the oral mucosa, and/or coat an area not readily accessible to fingertips. Topical treatment of mucosal membranes other than those in the oral cavity was not proposed in U.S. Pat. No. 5,362,737.

### SUMMARY OF THE INVENTION

It is the object of the present invention to provide a convenient and effective dosage form for amlexanox in the treatment of mucocutaneous disorders which allows for an effective dose of the pharmaceutical to be applied to the lesion(s) over an extended period. This is especially important for diseases and conditions in which a wide area of mucosa requires treatment, and/or when the application of the pharmaceutical to the lesion may involve more than simple use of a finger tip.

In order that mucocutaneous disorders are treated effectively by topical application of amlexanox, it is required that the lesion be in contact with an effective quantity of amlexanox for the period of time required to derive benefit. To grant such benefit, this invention provides for a stable solution of amlexanox in a mucoadhesive, viscous liquid. The liquid can readily be applied to the affected region of the mucosa by methods known in the art, while the high viscosity and mucoadhesion will cause amlexanox to remain in contact with the lesion for extended periods. The formulations of the present invention may be applied to treat mucocutaneous lesions in a variety of body compartments, including, but not limited to, the oral cavity, the nasal cavity, the esophagus, the rectum, the lower intestinal tract, the bladder, and the vagina.

The present invention involves a formulation usable in the treatment of ulcerative, inflammatory and erosive disorders of a mucosal membrane. The formulation comprises an effective amount of a mucoadhesive and amlexanox. The mucosal membranes include those of the oral cavity, the nasal cavity, the gastrointestinal tract, respiratory system, vaginal system, and bladder. The mucoadhesive is a water-soluble substance that adheres to mucosa. Such mucoadhesives may be water-soluble or water miscible vinyl polymers such as Carbopol, or water-soluble polyacrylic acid, cross-linked co-polymers of acrylic acid, carboxymethylcellulose, hydroxyalkylcellulose, dextran sulphate, dermatan sulphate and the like. A preferred mucoadhesive is the water-soluble or water miscible vinyl polymer Carbopol, most preferably Carbopol 971P. The formulation of the present invention may also contain a viscosity enhancer such as bentonite glycerin, providone, kaolin or tragacanth (most preferably glycerin). The formulation may contain an acid, a base or both utilized to maintain pH in the vicinity of neutrality (preferably 6.5-8.5). The acids or bases utilized to control the pH of the solution most preferably include sodium hydroxide, potassium hydroxide, phosphoric acid and citric acid. The formulation of the present invention may further comprise a preservative such as, e.g., benzyl alcohol, benzoate salts, phenoxyethanol, methylparaben and propylparaben. The formulation of the present invention may also contain a humectant. This humectant is generally from the group consisting of cholesterol, fatty acids, glycerine, lauric acid, magnesium stearate, pentaerythritol, and propylene glycol. The formulation of the present invention an emulsifying agent. A preferred emulsifying agent is a polysorbate 60. A pharmaceutically-acceptable flavoring agent or sweetener may also be added to the formulation of the present invention. Such sweeteners are most preferably saccharin, glycerin, simple syrup, or sorbitol. The formulation of the present invention may also contain an organic solvent, e.g., to further solubilize or stabilize other ingredients. An antioxidant is also a helpful ingredient of the present inventive formulation.

The mucoadhesive is generally at a concentration of from about 0.10 w/w % to about 3.0 w/w %. The concentration of the preferred mucoadhesive, Carbopol 971P is about 0.30 w/w % to about 0.60 w/w % (most preferably 0.30 to about 0.40 w/w %). The formulation of the present invention may also include a coloring agent as desired. A buffering agent useful to help stabilize the pH at desired levels is also an important option for the formulation of the present invention.

The amlexanox of the present inventive formulation is generally between about 0.05 w/w % and about 2.5 w/w %. A preferred range of amlexanox concentration is from about 0.1 w/w % to about 2.5 w/w %. The most preferred amlexanox concentration is from about 0.25 w/w % to about 1.0 w/w %.

The present invention involves the use of these formulations for the treatment inflammatory and erosive disorders of a mucosal membrane. In such cases, the formulation of the present invention is placed into contact with the inflamed or erosive mucosal membrane regions. This may be periodically repeated as desired until the disorder has been remedied.

### BRIEF DESCRIPTION OF THE DRAWINGS

FIG. 1 shows a cross-sectional view of the in vitro flow cell utilized to test formulations of the present invention.

FIG. 1A shows an overhead view of the in vitro flow cell utilized to test formulations of the present invention.

FIG. 2 shows a cross-sectional view of the in vitro flow cell of the present invention along with the connections and pumps for measuring amlexanox passage through the membrane or wash away from the membrane.

FIG. 3A describes the washing away of amlexanox from a membrane coated with a mucoadhesive (•) formulation of the present invention and the non-mucoadhesive (▪) mixture of amlexanox described in U.S. Pat. No. 5,362,787.

FIG. 3B describes the diffusion of amlexanox across the dialysis membrane 6 when applied in the presence (•) or absence (▪) of the mucoadhesive.

FIG. 4A describes amlexanox washed away from a dialysis membrane coated with a mucoadhesive (•) amlexanox preparation or a non-mucoadhesive but viscous (▪) preparation.

FIG. 4B describes amlexanox diffused across a dialysis membrane when applied in the presence of a mucoadhesive (•) material or non-mucoadhesive but viscous (▪) material.

FIG. 5 describes amlexanox washed away from an agar slab when applied in a viscous, mucoadhesive formulation (♦), a viscous, non-mucoadhesive formulation (▪), and a non-viscous, non-mucoadhesive formulation (▴).

### DETAILED DESCRIPTION OF THE INVENTION

The present invention includes the treatment of disorders of mucous membranes that, unlike those amenable to treatment utilizing the compositions claimed in U.S. Pat. No. 5,632,737, do not present as discrete, localized lesions in the oral cavity. The mucous membranes which may be treated by the compositions described in this patent include, but are not limited to, those in the oral cavity, the nasal cavity, the gastrointestinal and respiratory tracts, the vagina, and the bladder. Inflammatory, erosive, and/or ulcerative diseases which can be treated by topical application of the compositions described in this patent include, but are not limited to, aphthous ulcers, Behcet syndrome, chemical or radiation-induced cystitis, erythema multiforme, esophagitis, graft-versus-host disease, interstitial cystitis, mucositis, oral lichen planus, the pemphigoid group of diseases, pemphigus, and radiation proctitis. In conditions such as aphthous ulcers, chemical or radiation-induced cystitis, mucositis, and radiation proctitis, when the onset of the inflammatory, erosive, and/or ulcerative condition may be forecast (for example, by prodromal sensations in the case of aphthous ulcers, and by initiation of chemotherapy and/or radiation therapy in the treatment of cancer), the compositions of this invention might be applied prior to the formation of lesions to prevent or delay the onset of inflammatory, erosive, and/or ulcerative lesions.

The prevention and/or treatment of the above conditions utilizes the pharmaceutically active compound amlexanox (2-amino-7-(1-methylethyl)-5-oxo-5H[1]benzopyrano[2,3-b]pyridine-3-carboxylic acid) in a stable, mucoadhesive, viscous solution formulation. This solution is applied to the mucous membrane. The properties of the formulation are such that the solution stays in place for a prolonged period, delivering the drug to the lesion for an effective period.

Aqueous solutions of pharmaceutically-active compounds are well known in the art as convenient drug delivery formulations. Such formulations are most useful for oral delivery, when the solution is swallowed, and the drug is presented to the stomach and gastrointestinal tract in a form which is amenable to rapid absorption. Aqueous solutions are also used deliver drugs to mucosal tissue. In general, aqueous solutions used to deliver pharmaceuticals tend to be non-viscous and non-mucoadhesive. For oral delivery, this property is desirable, as it minimizes the amount of drug which is retained in the oral cavity and esophagus, while maximizing that delivered to the stomach and gastrointestinal tract.

For topical treatment of mucosal membranes, aqueous solutions of pharmaceutically-active compounds offer the advantage over other dosage forms in that a wide area of the mucosa can be readily covered with the solution, which is of benefit if the area to be treated is not a small, discrete region. Also, mucosa not readily accessible can be treated using aqueous solutions of pharmaceutically-active compounds and simple methods of application. However, formulations which are non-mucoadhesive and non-viscous are less than ideal for delivery of drugs to mucosal surfaces. Such solutions will be rapidly removed from the area being treated, for example, because the liquid flows from the site of application under the influence of gravity, and/or because the natural secretions of mucosal membranes carry the solution from the site of application.

An aqueous formulation of this invention is a stable, viscous, mucoadhesive liquid compositions containing amlexanox for the topical treatment of diseases and disorders of mucosal membranes. Neither high viscosity nor mucoadhesion alone confers ideal properties. A viscous but non-mucoadhesive liquid will not be held in place on the mucosal surface, instead a non-mucoadhesive solution will readily be lost from the point of application, for example, under the influence of gravity, and/or through natural movements of the membrane and surrounding structures. With an aqueous liquid formulation that is mucoadhesive but has low viscosity, a thin layer of the liquid which is adjacent to the mucosa will be held in place, but the bulk of the liquid could rapidly flow from the site of application under the influence of gravity and/or be readily removed by the natural secretions of mucosal membranes. In a mucoadhesive, viscous liquid formulation, the liquid will adhere to the mucosa, while the high viscosity of the liquid will reduce the rate of removal of the bulk of the liquid from the site of application.

Mucoadhesive formulations are well known in the art. However, known formulations intended for extended drug delivery to (or close to) the site of application of a mucoadhesive formulation are either solids or semi-solids. Examples of the former include tablets, powders, lozenges, and films. Examples of mucoadhesive semi-solids include gels, pastes, ointments, and creams. Few, if any, examples are known in which a mucoadhesive viscous liquid is used as a drug delivery system.

The formulations of the current invention are viscous but free-flowing (i.e. not gels). The ability to flow freely is necessary in order to readily coat either a selected region or a wide area of the affected mucosal membrane, and to coat mucosal membranes not readily accessible to simple application. The solutions of the current invention will preferably have viscosities in the range of 100-600 cps.

The stable, viscous, mucoadhesive liquid amlexanox formulations of the current invention may be applied to mucosal membranes for the prevention and/or treatment of disorders or lesions of these membranes. The liquid may be applied to the following mucosal surfaces; the oral cavity, the nasal cavity, the gastrointestinal and respiratory tracts, the vagina, and/or the bladder. The formulations of the current invention may also be applied to other mucous membranes for the prevention and treatment of disorders and diseases. The following methods of application are described to exemplify how the formulations might be applied. Other methods known in the art for the delivery of liquids to body compartments might also be used.

For treatment of disorders, lesions, and diseases of the oral cavity, the stable, viscous, mucoadhesive liquid amlexanox formulations of the current invention may be taken by mouth and distributed throughout the oral cavity by a swishing action, or by the patient adopting a slow circulating movement the head. Excess solution can either be swallowed or expelled.

For treatment of disorders and diseases of the esophagus, the stable, viscous, mucoadhesive liquid amlexanox formulations of the current invention can be swallowed with minimal contact of the oral cavity, administered by gavage, or administered by spraying the liquid into the throat. For treatment of disorders and diseases of the nasal cavity, the stable, viscous, mucoadhesive liquid amlexanox formulations of the current invention can be delivered as droplets or by spraying the liquid into the nose. For treatment of disorders and diseases of the bladder, the stable, viscous, mucoadhesive liquid amlexanox formulations of the current invention can be delivered by intra vesical administration, For treatment of disorders and diseases of the rectum and the large intestine, the stable, viscous, mucoadhesive liquid amlexanox formulations of the current invention can be administered by catheter or enema.

Other methods to apply the stable, viscous, mucoadhesive liquid amlexanox formulations of the current invention to mucosal tissues will be obvious to those skilled in the art.

In addition to the requirements of mucoadhesion and viscosity as described above, it is a requirement, for use of the formulation as a pharmaceutical, that the liquid solution of amlexanox be stable, such that it can be stored at ambient temperatures for many months or years, even when subjected to brief periods of elevated or depressed temperatures, without physical or chemical degradation of the formulation. It is further desirable to formulate the product without use of any organic solvents, the presence of which might irritate the mucosal lesions being treated. Furthermore, it is desirable to formulate the liquid solution of amlexanox only using excipients which are accepted by all major pharmaceutical regulating authorities as safe.

Amlexanox is a lipophilic molecule that is poorly water soluble. The molecule contains a free carboxylate group, and the water solubility of amlexanox can be increased by raising the pH of the dissolution solution. However, caution is necessary using this approach as degradation of amlexanox may take place at high pH. Additionally, amlexanox can degrade slowly in aqueous solution under certain conditions if the solution pH is greater than about 8.0. Thus the formulation of a viscous, mucoadhesive solution of amlexanox which is also stable represented a major challenge.

According to this invention, aqueous solutions of amlexanox which are viscous and mucoadhesive will contain the following classes of excipients:

a). A linear or cross-linked polymer which provides mucoadhesion. Such polymers include (but are not limited to) polyacrylic acid, a cross-linked homopolymer based on acrylic acid, a cross-linked copolymer based on acrylic acid, carboxymethylcellulose, hydroxyalkylcellulose, dextran sulfate, dermatan sulfate, and chitosan. Other mucoadhesive polymers are well-known to those skilled in the art. The aqueous formulation of amlexanox can contain a single mucoadhesive component, or mixtures thereof. The preferred mucoadhesive polymers are a cross-linked homopolymers and copolymers based on acrylic acid, especially the Carbopol and Noveon polymers supplied by B.F. Goodrich; most preferred is Carbopol 971P.

b). Viscosity enhancement is provided by one or more of the above mentioned mucoadhesive polymers alone or in combination with agar, bentonite, glycerin, providone, kaolin, and/or tragacanth. Most preferred are Carbopol 971P in combination with glycerin.

C). The pH of the solution is adjusted to the final desired pH with any pharmaceutically accepted acid or base. Most preferred are sodium or potassium hydroxide, phosphoric acid, or citric acid. A final pH of 6.5 to 8.5 is preferred (7.0 to 8.0 being most preferred).

d). To prevent or inhibit microbial growth in the formulation during storage, it is desirable to include a preservative. Preservatives well-known in the art include benzyl alcohol, benzoate salts, phenoxyethanol, methylparaben, and propylparaben. Benzyl alcohol is the preferred preservative.

e). A humectant is desirable to provide a pleasant mouth-feel in oral applications. Humectants known in the art include cholesterol, fatty acids, glycerin, lauric acid, magnesium stearate, pentaerythritol, and propylene glycol. Glycerin is preferred.

f). An emulsifying agent might be necessary, for example to ensure complete dissolution of all excipients, especially hydrophobic components such as benzyl alcohol. Many emulsifiers are known in the art. The preferred emulsifier is polysorbate 60

g). For oral applications, it may be desirable to add a pharmaceutically acceptable flavoring agent and/or sweetener. Compounds such as saccharin, glycerin, simple syrup, and sorbitol are useful as sweeteners. Saccharin is preferred.

It may be desirable to include other ingredients; for example a pharmaceutically acceptable organic solvent, a buffering agent, an antioxidant, an antimicrobial agent, and /or a coloring agent. The exact formulation of the above ingredients, and the method of manufacture, will be apparent to those skilled in the art. A number of texts provide assistance in the design and manufacture of pharmaceutical formulations, including Remington's Pharmaceutical Sciences, Mack Publishing Company Co., Easton, Pa., and Pharmaceutical dosage forms and drug delivery, Ansel et al, 1995, Williams and Wilkins, Malvern, Pa.

The % amlexanox in these solutions can be about 0.05% to about 5.0% w/w. It is preferred that the amlexanox concentration be 0.1% to 2.5 w/w %, and most preferred that the amlexanox concentration be 0.25% to 1.0 w/w %.

In the development of a stable, mucoadhesive, viscous aqueous solution of amlexanox which is suitable for pharmaceutical applications, it became evident that not all combinations of the excipients detailed above would provide a solution with all the necessary characteristics. In particular, the choice of the mucoadhesive polymer appeared to be important. It is considered that Carbopol 971P, in addition to providing the mucoadhesive and viscosity properties of the preferred composition, might also stabilize the solution of amlexanox. Other polymers, such as polyacrylic acids, a cross-linked homopolymers based on acrylic acid, cross-linked copolymers based on acrylic acid, carboxymethylcellulose, hydroxyalkylcellulose, dextran sulfate, or dermatan sulfate, including Carbopol 974P and Carbopol 934P, might also fulfill this function by making minor formulation adjustments that are known to those skilled in the art. Carbopol the trade name given to a series of water-miscible or water-soluble cross-linked vinyl polymers made by the B. F. Goodrich Chemical Co. The preferred thickening/mucoadhesive ingredient in our aqueous solution is Carbopol 971P. The Carbopol series of pharmaceutical excipients are well known for their ability to thicken and provide mucoadhesive properties. This is best exemplified by the technical “bulletins” provided by the manufacturer, B. F. Goodrich. There are 19 bulletins on the Carbopol series; the most relevant are are Bulletin 11—Thickening Procedures, and Bulletin 16—Bioadhesion.

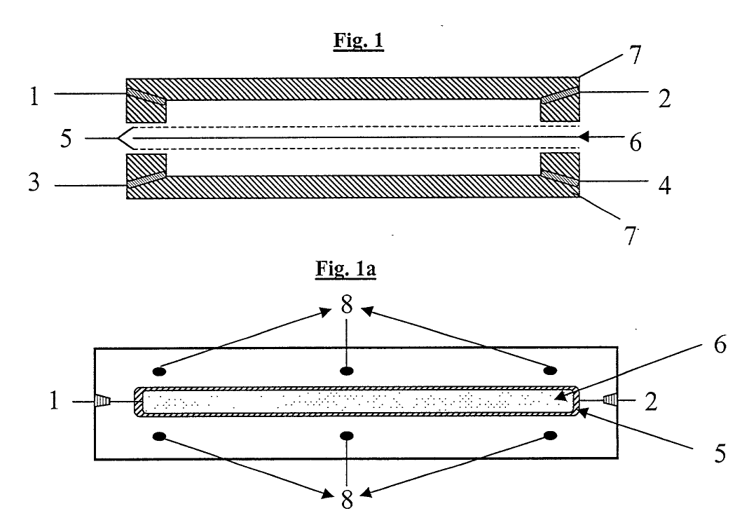

An in vitro model was developed to demonstrate the enhanced delivery of amlexanox to the surface of mucosal membranes. Referring to FIG. 1 and FIG. 1a, there are two identical clear polycarbonate blocks 7 which are 1.2 cm×4.7 cm×11.5 cm having a 0.2 cm×1.0 cm×10.1 cm flat-bottomed groove 9. Each opposing end of the flat-bottomed groove 9 has a 10/32″ port 1-4 for connecting tubing for the flow of artificial saliva and/or adding sample solutions. Separating the two identical clear polycarbonate blocks 7 is a Spectra/Por 4 dialysis membrane 6 which has a layer of Gore-Tex® GR gasket sheeting on both sides to prevent leakage. The two identical halves of polycarbonate 7 are joined together to form a watertight flow cell with six bolts and six wing nuts through perforations 8.

TABLE 1
Pharmacology of Amlexanox

Potent ant-allergic agent demonstrated in a variety of in vivo models
Potent inhibitor of the release of inflammatory mediators from mast cells
Antigen-antibody induced release
Ionophore induced release
Potent inhibitor of generation of 5- and 12-lipoxygenase products in
whole tissue studies.
Inhibits formation of leukotrienes, in vitro
Does not inhibit generation of cyclo-oxygenase products in whole
tissue studies
Potent inhibitor of cyclic nucleotide phosphodiesterase
Inhibits (in vivo) brochoconstriction inducted by:
Has no effect on bronchoconstriction induced by histamine or
acetylcholine
LTD 4 (leukotriene)
PAF
Antigen
Weak anti-inflammatory activity in vivo

### EXAMPLES Example 1

### Preparation of Viscous, Mucoadhesive Aqueous Composition Containing Amlexanox

A viscous, mucoadhesive aqueous solution of amlexanox was formulated by adding Carbopol® 971P NF to water using an appropriate mixing apparatus (Master Servodyne® mixer with high-lift blade rotating at 200-300 rpm) to give a clear solution. An aqueous solution of potassium hydroxide was added with stirring to give a clear gel. An aqueous solution of potassium hydroxide, citric acid, saccharin sodium, amlexanox, phosphoric acid and glycerin was added with stirring to give a clear solution. A solution of benzyl alcohol and polysorbate 60 was added with stirring to give a clear solution. The pH was adjusted to 7.0-7.8 with an aqueous solution of phosphoric acid. The resulting product was mixed further for 30 minutes. The formulation of the product is set out, below in Table 2.

TABLE 2

| Ingredients | Weight Percent |
|---|---|
| purified water | 91.26 |
| potassium hydroxide | 0.60 |
| benzyl alcohol | 1.50 |
| polysorbate 60 | 0.05 |
| Carbopol ® 971P | 0.35 |
| phosphoric acid | 0.13 |
| citric acid | 0.05 |
| saccharin sodium | 0.40 |
| amlexanox | 0.50 |
| glycerin | 5.20 |

### Example 2

### Preparation of a Second Viscous, Mucoadhesive Aqueous Composition Containing Amlexanox

The method set out in Example 1, substituting Carbopol® 974P for Carbopol® 971P, was followed to provide a viscous, mucoadhesive aqueous composition of the following formula (see Table3).

TABLE 3

| Ingredients | Weight Percent |
|---|---|
| purified water | 90.73 |
| potassium hydroxide | 0.46 |
| benzyl alcohol | 1.55 |
| polysorbate 60 | 0.05 |
| Carbopol ® 974P | 0.52 |
| phosphoric acid | 0.04 |
| citric acid | 0.05 |
| saccharin sodium | 0.42 |
| amlexanox | 1.04 |
| glycerin | 5.15 |

### Example 3

### Preparation of a Third Viscous, Mucoadhesive Aqueous Composition Containing Amlexanox

The method set out in Example 1 was followed to provide a viscous, mucoadhesive aqueous composition of the following formula as seen in Table 4.

TABLE 4

| Ingredients | Weight Percent |
|---|---|
| purified water | 90.90 |
| potassium hydroxide | 0.60 |
| benzyl alcohol | 1.51 |
| polysorbate 60 | 0.05 |
| Carbopol ® 971P | 0.35 |
| phosphoric acid | 0.03 |
| citric acid | 0.05 |
| saccharin sodium | 0.40 |
| amlexanox | 1.00 |
| glycerin | 5.10 |

### Example 4

### Preparation of a Fourth Viscous, Mucoadhesive Aqueous Composition Containing Amlexanox

The method set out in Example 1 was followed to provide a viscous, mucoadhesive aqueous composition of the following formula as seen in Table 5.

TABLE 5

| Ingredients | Weight Percent |
|---|---|
| purified water | 91.26 |
| potassium hydroxide | 0.60 |
| benzyl alcohol | 1.50 |
| polysorbate 60 | 0.01 |
| Carbopol ® 971P | 0.35 |
| phosphoric acid | 0.13 |
| citric acid | 0.05 |
| saccharin sodium | 0.40 |
| amlexanox | 0.50 |
| glycerin | 5.20 |

### Example 5

### Preparation of a Fifth Viscous, Mucoadhesive Aqueous Composition Containing Amlexanox

The method set out in Example 1 was followed to provide a viscous, mucoadhesive aqueous composition of the following formula as seem in Table 6.

TABLE 6

| Ingredients | Weight Percent |
|---|---|
| purified water | 91.86 |
| potassium hydroxide | 0.60 |
| benzyl alcohol | 1.50 |
| polysorbate 60 | 0.05 |
| Carbopol ® 971P | 0.36 |
| phosphoric acid | 0.18 |
| citric acid | 0.05 |
| saccharin sodium | 0.40 |
| amlexanox | 0.50 |
| glycerin | 4.90 |

### Example 6

### Preparation of a Sixth Viscous Mucoadhesive Aqueous Composition Containing Amlexanox

The method set out in Example 1 was followed, except that an aqueous solution of flavor was added with stirring to give a clear solution prior to pH adjustment with phosphoric acid solution. This gave a viscous, mucoadhesive aqueous composition of the following formula as seen in Table 7.

TABLE 7

| Ingredients | Weight Percent |
|---|---|
| purified water | 91.25 |
| potassium hydroxide | 0.60 |
| benzyl alcohol | 1.50 |
| polysorbate 60 | 0.05 |
| Carbopol ® 971P | 0.35 |
| phosphoric acid | 0.19 |
| citric acid | 0.05 |
| saccharin sodium | 0.40 |
| amlexanox | 0.50 |
| glycerin | 5.06 |
| natural orange flavor | 0.05 |

### Example 7

### Preparation of a Seventh Viscous, Mucoadhesive Aqueous Composition Containing Amlexanox

The method set out in Example 6 was followed to provide a viscous, mucoadhesive aqueous composition of the following formula as seen in Table 8.

TABLE 8

| Ingredients | Weight Percent |
|---|---|
| purified water | 91.62 |
| potassium hydroxide | 0.32 |
| benzyl alcohol | 1.52 |
| polysorbate 60 | 0.05 |
| Carbopol ® 971P | 0.36 |
| phosphoric acid | 0.22 |
| citric acid | 0.05 |
| saccharin sodium | 0.40 |
| amlexanox | 0.50 |
| glycerin | 4.95 |

### Example 8

### Preparation of an Eighth Viscous, Mucoadhesive Aqueous Composition Containing Amlexanox

The method set out in Example 6 was followed to provide a viscous, mucoadhesive aqueous composition of the following formula as seen in Table 9.

TABLE 9

| Ingredients | Weight Percent |
|---|---|
| purified water | 91.11 |
| potassium hydroxide | 0.61 |
| benzyl alcohol | 1.53 |
| polysorbate 60 | 0.05 |
| Carbopol ® 971P | 0.36 |
| 0.5% phosphoric acid | 0.15 |
| citric acid, anhydrous | 0.05 |
| saccharin sodium | 0.41 |
| amlexanox | 0.51 |
| glycerin | 5.17 |
| natural orange flavor | 0.05 |

### Example 9

### Preparation of a Viscous, Mucoadhesive Aqueous Composition

The method set out in Example 1 was followed, except that amlexanox was excluded, to provide the viscous, mucoadhesive aqueous composition as seen in Table 10. The formulation contains a larger quantity of mucoadhesive polymer compared with previous examples:

TABLE 10

| | |
|---|---|
| Ingredients | Weight Percent |

| | | |
|---|---|---|
| purified water | | 92.04 |
| potassium hydroxide | c.a. | 0.61 |
| benzyl alcohol | | 1.45 |
| polysorbate 60 | | 0.05 |
| Carbopol ® 971P | | 0.48 |
| 0.5% phosphoric acid | | 0.11 |
| citric acid | c.a. | 0.05 |
| saccharin sodium | | 0.39 |
| amlexanox | | 0.0 |
| glycerin | | 4.83 |

### Example 10

### High Performance Liquid Chromatographic Assay for the Amlexanox

The following HPLC parameters were employed in performing an assay for amlexanox:

Phenomenex, Prodigy, 5 μm ODS (2), 150 mm×4.6 mm

Mobile Phase: 25% THF/75% 10 mM phosphate buffer, pH 8.0

Flow Rate: 1.0 mL/min

Injection Volume: 10 μL

Detector: UV@ 244 nm

### Example 11

### Demonstration of Drug Delivery from Liquid Formulations in an in vitro Model of a Mucosal Surface

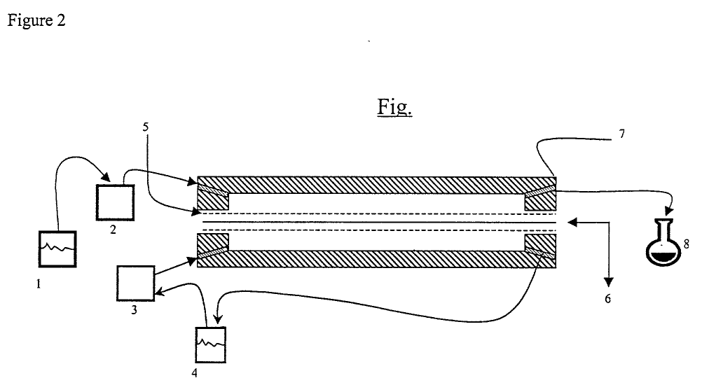

Referring to FIG. 2, the device described in FIG. 1 and 1A is attached to LC pumps 2, 3 using 10/32″ ferules and large diameter HPLC tubing. One half of the polycarbonate block 7 has a reservoir 1 that provides a continuous flow artificial saliva across the Spectra/Por4 dialysis membrane 6 and is eluted into a vessel 8. The opposing half of the polycarbonate block 7 is connected to a LC pump 3 which has a reservoir 4 of artificial saliva that constantly recirculates across the dialysis membrane 6.

In two separate experiments, the composition of Example 8 was compared with an aqueous rinse formulation of the following formulation:

Amlexanox 0.50; trolamine 1.01, ethanol 12.80, glycerin 3.00, water 82.26, sodium citrate dihydrate 0.21, and citric acid 0.20 (all % w/w). This formulation is described in Example 3 of U.S. Pat. No. 5,362,737. 2.9-3.1 mL of the aqueous formulation of amlexanox is applied to the dialysis membrane of the in vitro system, and the flow of artificial saliva was initiated at a rate of 1.0 mL/min. mL samples were withdrawn from the reservoirs 4 and 6 and samples were assayed for amlexanox content using the HPLC assay described in Example 10.

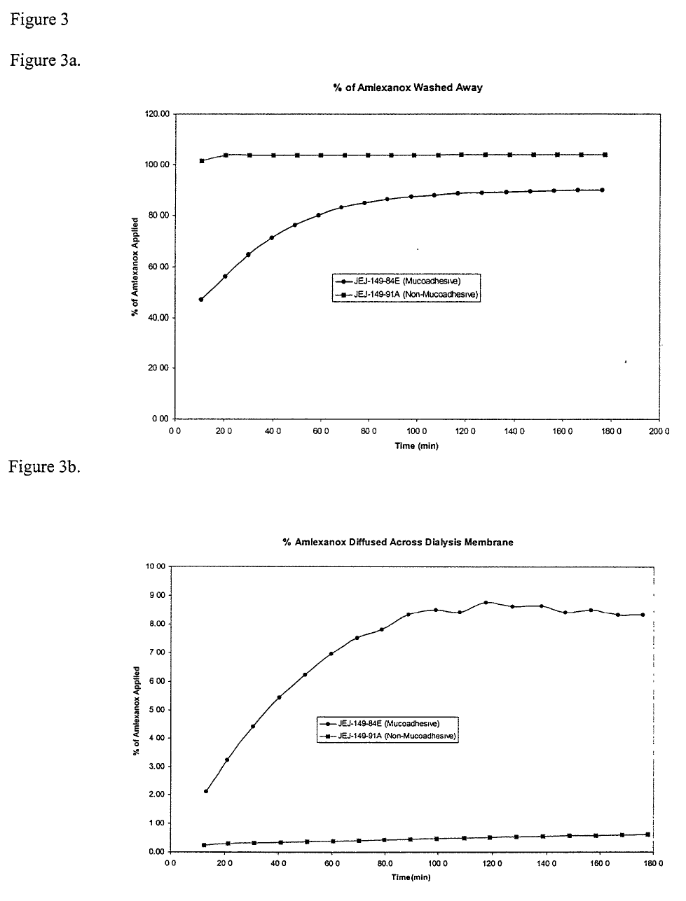

The results of this study are shown in FIG. 3A and 3B. As can be seen in FIG. 3a, much less amlexanox is washed away in the simulation of saliva flow in the case of the viscous, mucoadhesive solution which is the subject of this invention compared with the rinse which is described in U.S. Pat. No. 5,362,737. As a result, much more amlexanox (the data indicates a ten-fold difference) is delivered to the membrane and transported to the receiver vessel in the case of the viscous, mucoadhesive rinse which is the subject of this invention compared with the rinse which is described in U.S. Pat. No. 5,362,737.

### Example 12

### Comparison of Drug Delivery from a Viscous Mucoadhesive Solution and a Viscous non-mucoadhesive Solution.

The study described in Example 11 was repeated using the composition of Example 8 (as the viscous mucoadhesive solution) and the following viscous, non-mucoadhesive composition:

| Ingredients | % wt/wt |
|---|---|
| Water | 81.01 |
| Citric Acid | 0.05 |
| Sodium Saccharin | 0.40 |
| 10% KOH | 1.78 |
| Amlexanox | 0.50 |
| Glycerin | 5.01 |
| Benzyl Alcohol | 1.50 |
| Polysorbate 60 | 0.05 |
| 2.51% Phosphoric Acid | 0.49 |
| Polyvinyl Pyrrolidone | 9.21 |
| Final pH | 7.841 |

The viscosities of the two solutions were measured using a Brookfield DV-II+ viscometer at 25±1° C. The viscosity of the non-mucoadhesive solution was 207.5 cP, and the viscosity of the mucoadhesive solution was 216.1 cP.

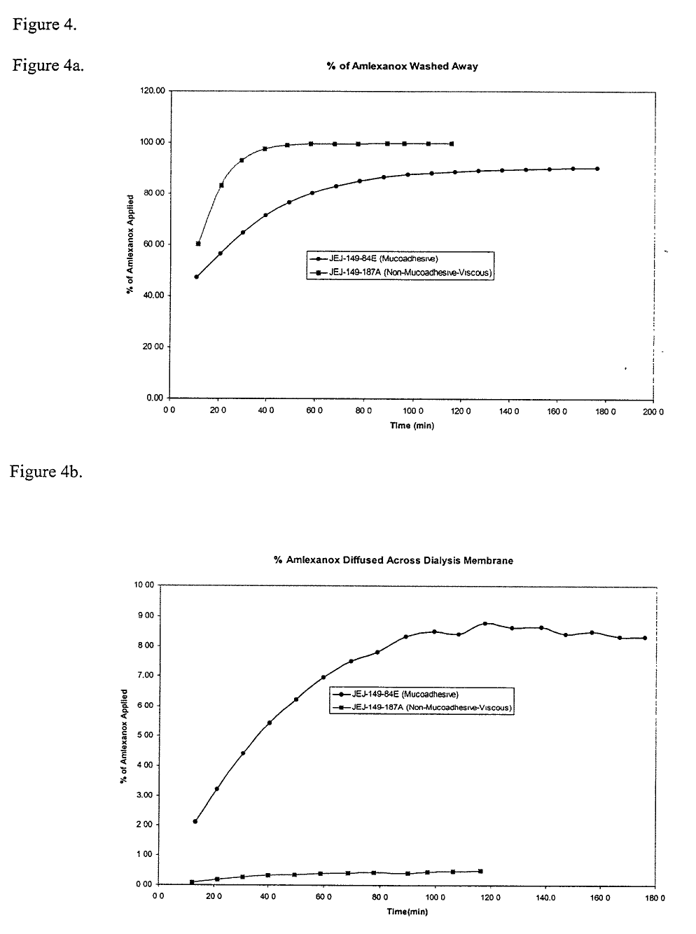

The graphs shown in FIG. 4 display the results of this study. The upper graph shows the amount of amlexanox which is washed away in the simulated saliva flow. As can be seen, much less amlexanox is washed away in the case of the viscous, mucoadhesive solution which is the subject of this invention compared with the viscous, non-mucoadhesive solution. As a result, much more amlexanox is delivered to the membrane and transported to the receiver vessel in the case of the viscous, mucoadhesive rinse compared with the viscous, non-mucoadhesive solution, as shown in the lower graph.

### Example 13

### Comparison of the Rate of Drug Washed away from a 1.5% Agar Slab from a Viscous Mucoadhesive solution, a Viscous Non-mucoadhesive Solution, and a Non-viscous Non-mucoadhesive Solution

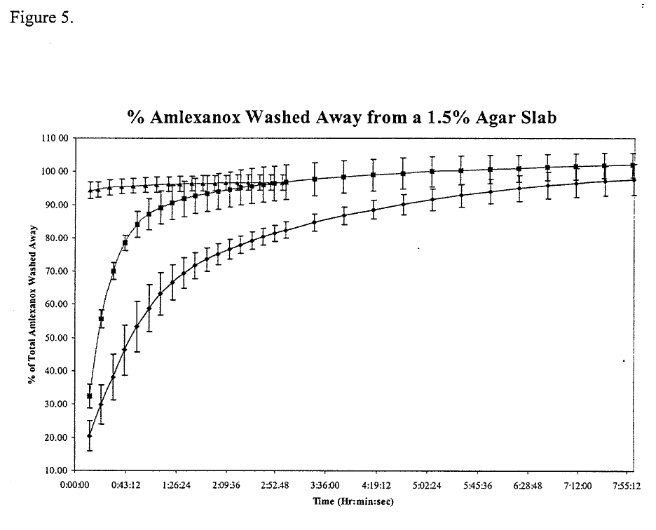

A slab of agar can serve as a good model system of the mucosa in in vitro mucoadhesion studies (D. J. Dobrozso et al., Int. J. Pharm. 189 (1999) 81-89). The in vitro model of a mucosal surface described in Example 11 was used in conjunction with an agar slab to measure the rate of release of amlexanox from the slab into a stream of simulated saliva flowing over the preparation. The 1.5% Agar preparation was made by adding 0.15 grams of agar to 9.85 grams of distilled water. The mixture was heated and stirred continuously until dissolved. The bottom chamber of the flow cell was filled with hot 1.5% agar solution. A flat piece of polycarbonate was placed on top of the hot 1.5% agar solution for 10 minutes. Excess agar gel was removed with the edge of the flat piece of polycarbonate. (Note: The agar slab was used within 30 minutes of gelling).Construction of the flow cell was completed as described in example 11. Using a tared syringe, 1.0 grams of test amlexanox solution was added through the inlet port of the top half of the flow cell. The timer was started; after 30 seconds, the flow of artificial saliva a rate of 1.0 mL/minute was started through the inlet port of the top chamber of the flow cell. The eluent was collected in a 10 mL or 25 mL volumetric flask and the time for each aliquot was recorded. HPLC analyses for amlexanox content of the artificial saliva were conducted as described in example 11. FIG. 5 shows the result of comparing the viscous, mucoadhesive formulation of example 8 with the viscous, non-mucoadhesive formulation of example 12, and the non-viscous, non-mucoadhesive formulation in U.S. Pat No. 5,362,737, as described in example 11.

### Example 14

### Stability Study

The composition of Example 8 was subjected to a stability study. The clear solution was packaged in clear glass bottles which were sealed with white screw caps fitted with teflon liners. The bottles were divided into two groups. One group was stored in a stability chamber set at 25° C./60% relative humidity, while the second group was stored at 40° C./75% relative humidity. Bottles were examined at 0, 1, 2, 3, and 6 months for physical appearance (clarity of the solution), package integrity, amlexanox and benzyl alcohol contents, pH, and viscosity. At all times and under both conditions, no physical or chemical changes were noted.

What is claimed is:

1. A formulation useable in the treatment or prevention of ulcerative, inflammatory, and erosive diseases or disorders of a mucosal membrane, the formulation comprising effective amounts of a mucoadhesive and amlexanox.

2. The formulation of claim 1 defined further as stable, viscous, and mucoadhesive.

3. The formulation of claim 1 where said mucosal membrane includes oral cavity membrane, nasal cavity membrane, gastrointestinal membrane, respiratory tract membranes, vaginal membrane, or bladder membrane.

4. The formulation of claim 1 where said mucoadhesive is a linear or cross-linked polymer.

5. The formulation of claim I where said diseases or disorders include aphthous ulcers, oral lichen planus, oral mucous membrane contact dermatitis, sinusitis, rhinitis, cystitis, Behcet syndrome, graft-versus-host disease, the pemphigoid group of diseases, mucositis, esophagitis, and radiation proctitis.

6. The formulation of claim 4 where said linear or cross linked polymer is at least one of the group consisting of polyacrylic acid, a cross-linked copolymer based on acrylic acid, carboxymethylcellulose, hydroxyalkyl cellulose, dextran sulfate, dermatan sulfate, a water-soluble vinyl polymer, and chitosan.

7. The formulation of claim 4 where said linear or cross-linked polymer is about 0.10 w/w % to about 3.0 w/w %.

8. The formulation of claim 4 where said linear or cross-linked polymer is a Carbopol.

9. The formulation of claim 4 where said linear or cross-linked polymer is Carbopol 971P.

10. The formulation of claim 4 defined further as containing a viscosity enhancer.

11. The formulation of claim 10 where said viscosity enhancer is selected from the group consisting of agar, bentonite, glycerin, providone, kaoline, and tragacanth.

12. The formulation of claim 1 defined further as containing glycerin.

13. The formulation of claim 1 defined further as containing at least one of an acid and base.

14. The formulation of claim 13 where the acid or base is to maintain a pH of between about 6.5 and about 8.5.

15. The formulation of claim 13 where the acid or base is to maintain a pH of between about 7.0 and about 8.0.

16. The formulation of claim 13 where the acid or base is selected from the group consisting of sodium hydroxide, potassium hydroxide phosphoric acid and citric acid.

17. The formulation of claim 1 defined further as containing a preservative.

18. The formulation of claim 17 where the preservative is at least one selected from the group consisting of benzyl alcohol, benzoate salts, phenoxyethanol, methylparaben, and propylparaben.

19. The formulation of claim 1 defined further as containing a humectant.

20. The formulation of claim 19 where the humectant is selected from the group consisting of cholesterol, fatty acid, glycerin, lauric acid, magnesium stearate, pentaerythritol, and propylene glycol.

21. The formulation of claim 1 further defined as containing an emulsifying agent.

22. The formulation of claim 21 where said emulsifying agent is polysorbate 60.

23. The formulation of claim 1 further defined as containing a flavoring agent or sweetener.

24. The formulation of claim 23 where the sweetener is selected from the group consisting of saccharin, glycerin, simple syrup, and sorbitol.

25. The formulation of claim 1 defined further as containing an organic solvent.

26. The formulation of claim 1 defined further as containing an antioxidant.

27. The formulation of claim 1 defined further containing from about 0.30 w/w % to about 0.60 w/w % Carbopol 971P.

28. The formulation of claim 9 defined further is about 0.30 w/w % to about 0.40 w/w % Carbopol 971P.

29. The formulation of claim 1 defined further as containing a coloring agent.

30. The formulation of claim 1 defined further as containing a buffering agent.

31. The formulation of claim 1 where the amlexanox is from about 0.05 w/w % to about 2.5 w/w %.

32. The formulation of claim 1 where the amlexanox is from about 0.1 w/w % to about 2.5 w/w %.

33. The formulation of claim 1 where the amlexanox is from about 0.25 w/w % to about 1.0 w/w %.

34. A method of treating ulcerative, inflammatory, erosive diseases or disorders of the mucosal membrane comprising:
identifying an individual who has a disease or disorder of a mucosal membrane, or is at risk from developing a disease or disorder of a mucosal membrane;
obtaining a premixed formulation useable in the treatment or prevention of diseases or disorders of the mucosal membrane comprising effective amounts of mucoadhesive and amlexanox; and
administering an effective amount of said formulation topically to said disease or disorder of the mucosal membrane of said individual such that there is prevention or improvement.

35. The method of claim 34 where the mucoadhesive is a linear or cross-linked polymer.

36. The method of claim 35 where said linear or cross-linked polymer is about 0.10 w/w % to about 3.0 w/w %.

37. The method of claim 35 where the formulation is defined further as containing a viscosity enhancer.

38. The method of claim 37 where said viscosity enhancer is selected alone or in combination from the group consisting of agar, bentonite, glycerin, providone, kaoline, or tragacanth.

39. The method of claim 35 here said linear or cross-linked polymer is Carbopol 971P.

40. The method of claim 39 defined further as containing about 0.30 w/w % to about 0.60 w/w % Carbopol 971P.

41. The method of claim 39 defined further as containing about 0.30 w/w % to about 0.40 w/w % Carbopol 971P.

42. The method of claim 34 where said amlexanox is from about 0.05 w/w % to about 2.5 w/w %.

43. The method of claim 34 where said amlexanox is from about 0.1 w/w % to about 2.5 w/w %.

44. The method of claim 34 where said amlexanox is from from about 0.25 w/w % to about 1.0 w/w %.

45. The method of claim 34 where said diseases or disorders include aphthous ulcers, oral lichen planus, oral mucous membrane contact dermatitis, sinusitis, rhinitis, cystitis, Behcet syndrome, graft-versus-host disease, the pemphigoid group of diseases, mucositis, esophagitis, and radiation proctitis.

46. A method of preventing ulcerative, inflammatory, erosive diseases or disorders of the mucosal membrane comprising:
identifying an individual who has had or who is likely to have a disease or disorder of the mucosal membrane;
obtaining a premixed formulation useable in the treatment or prevention of diseases or disorders of the mucosal membrane comprising effective amounts of mucoadhesive and amlexanox;
administering an effective amount of said formulation topically to the areas of the mucosal membrane where said disease or disorder of the mucosal membrane of said individual is likely to occur.

47. The method of claim 46 where the mucoadhesive is a linear or cross-linked polymer.

48. The method of claim 47 where said linear or cross-linked polymer is about 0.10 w/w % to about 3.0 w/w %.

49. The method of claim 46 where the formulation is defined further as containing a viscosity-enhancer.

50. The method of claim 49 where said viscosity enhancer is selected alone or in combination from the group consisting of agar, bentonite, glycerin, providone, kaoline, and tragacanth.

51. The method of claim 47 where said linear or cross-linked polymer is Carbopol 971P.

52. The method of claim 51 defined further as containing from about 0.30 w/w % to about 0.60 w/w % Carbopol 971P.

53. The method of claim 51 defined further as containing from about 0.35 w/w % to about 0.36 w/w % Carbopol 971P.

54. The method of claim 46 where said amlexanox is from about 0.05 w/w % to about 2.5 w/w %.

55. The method of claim 46 where said amlexanox is from about 0.1 w/w % to about 2.5 w/w %.

56. The method of claim 46 where said amlexanox is from about 0.25 w/w % to 1.0 w/w %.

57. The formulation of claim 46 where said diseases or disorders include aphthous ulcers, oral lichen planus, oral mucous membrane contact dermatitis, sinusitis, rhinitis, cystitis, Behcet syndrome, graft-versus-host disease, the pemphigoid group of diseases, mucositis, esophagitis, and radiation proctitis.
